(19) United States
(12) Patent Application Publication
(10) Pub. No.: US 2004/0185564 A1
(43) Pub. Date: Sep. 23, 2004

(54) Biodegradable copolymer and nucleic acid delivery system

(76) Inventors: Guping Tang, Singapore (SG); Yuexia Ma, Singapore (SG); Shu Wang, Singapore (SG)

Correspondence Address: BIRCH STEWART KOLASCH & BIRCH, PO BOX 747, FALLS CHURCH, VA, 22040-0747, US

(21) Appl. No.: 10/761,202

(22) Filed: Jan. 22, 2004

(30) Foreign Application Priority Data
Jan. 23, 2003 (SG) 200300154-2

(51) Int. Cl. C12N 15/85; C08G 63/91; C08G 63/48

(52) U.S. Cl. 435/455; 525/54.2

(57) ABSTRACT

The present invention provides positively chargeable biodegradable copolymers composed of a low molecular weight polycationic copolymer polyethylenimine linked by cyclodextrin, a method for the copolymer synthesis and the method of using the copolymers for intracellular delivery of nucleic acid molecules. The copolymers comprise ester bonds for hydrolysis and positively charged amines for forming complexes with negatively charged nucleic acids. The copolymers of the invention are useful as delivery systems of therapeutic genetic materials for gene therapy.

### TECHNICAL FIELD

The present invention relates to biodegradable copolymers containing polyethylenimine that are capable of complexing with nucleic acid molecules.

### BACKGROUND

Gene therapy is emerging as a modern medical intervention for the treatment of a variety of disorders. Viral vectors and non-viral delivery systems are two principle means for carrying out gene therapy. Although many different viral vectors have been developed or proposed for gene therapy, there are concerns about safe use of viruses and their efficacy for human applications. Non-viral gene delivery systems based upon DNA/chemical complexes, usually plasmid DNA, have gained increasing attention for their potential in avoiding problems inherent in viral gene vectors. Ideal chemical gene delivery vehicles should be bio-absorbable, non-toxic, non-immunogenic, stable during storage and after administration, able to access target cells, and suitable for efficient gene expression.

Among non-viral vectors in use, the polycationic copolymer polyethylenimine (PEI) has shown high transfection efficiency both in vitro and in vivo. PEI has been used for many years in a diversity of processes in many different fields. PEI comes in two forms: linear and branched. The branched form is produced by cationic copolymerization from ethylenimine (aziridine) monomers via a chain-growth mechanism, with branch sites arising from specific interactions between two growing copolymer molecules. The basic unit of PEI has a backbone of two carbons followed by one nitrogen atom. The branched PEI contains 1°, 2° and 3° amines, each with the potential to be protonated. PEI and other effective copolymeric gene carriers currently available, such as dendrimers, are not degradable.

Dependent on molecular weight, structure and doses, PEI can be toxic to certain cells. It has been reported that branched PEI with molecular weight (MW) of 25,000 or greater displayed relatively high cytotoxicity, possibly because of forming large aggregates on the surface of cells. Low molecular weight PEIs (<1,800) displayed much less toxicity but almost no transfection efficiency.

Cyclodextrins (CyDs) are cyclic (α-1,4)-linked oligosaccharides of α-D-glucopyranose containing a hydrophobic central cavity and hydrophilic outer surface. The most common CyDs are α-, β- and γ-CyDs, which consist of six, seven and eight D-glucopyranose units, respectively. The lack of toxicity and immunogenicity of these molecules has been well documented and are able to form inclusion complexes with a variety of guest molecules in solution and in solid state. Although CyDs themselves are not effective gene carriers, they can act as a viral dispersant, resulting in an increase in adenoviral transduction in human colon adenocarcinoma Caco-2 cells by enhancing both viral binding and internalization. CyDs can disrupt biological membranes by complexation with phospholipids and cholesterols. These effects have been suggested to assist cell take-up of polyamidoamine dendrimer/DNA particles and intracellular trafficking of DNA molecules.

The present inventors have used cyclodextrins to cross-link low molecular weight PEI polymers to increase the number of amine groups per each copolymer molecule, thus enhancing the capability of the PEI polymers in binding, neutralizing and condensing DNA, yet retaining their low toxicity. The biodegradable ester bonds are introduced during the activation of CyDs and the conjugation of activated CyD with PEI. By combining PEI and CyD molecules together, the resulting copolymers have been surprisingly found to act in a synergistic manner as a safe and effective delivery system for nucleic acids.

The present inventors have developed new copolymers capable of associating with nucleic acid molecules in plasmid form which are particularly suitable for gene therapeutic uses.

### SUMMARY OF INVENTION

In a first aspect, the present invention provides a biodegradable copolymer suitable for delivering a nucleic acid molecule to a cell, the copolymer comprising low molecular weight polyethylenimine (PEI) cross-linked by a cyclodextrin (CyD).

In a second aspect, the present invention provides a method for synthesizing a biodegradable copolymer comprising the steps of:

(a) treating cyclodextrin with an agent to form a modified or activated cyclodextrin; and

(b) adding the modified or activated cyclodextrin to a low molecular weight polyethylenimine to form a mixture and treating the mixture under suitable conditions to form a biodegradable copolymer comprising polyethylenimine linked by cyclodextrin.

Typically, a polycondensation reaction occurs between a modified or activated cyclodextrin and a low molecular weight polyethylenimine. Hydroxyl groups in cyclodextrin are coupled to anime groups in polyethylenimine with the result of a one-carbon spacer.

In a third aspect, the present invention provides biodegradable copolymer synthesized by the method according to the second aspect of the present invention.

In a fourth aspect, the present invention provides a method for delivering a nucleic acid molecule to a cell, the method comprising forming a complex between a biodegradable copolymer according to the first or third aspects of the present invention and a nucleic acid molecule and exposing the cell to the copolymer/nucleic acid molecule complex such that the complex is internalized by the cell and the nucleic acid molecule is released in the cell.

In a fifth aspect, the present invention provides use of a biodegradable copolymer according to the first or third aspects of the present invention to deliver a nucleic acid molecule to a cell.

In a sixth aspect, the present invention provides use of a biodegradable copolymer according to the first or third aspects of the present invention in the manufacture of a medicament for the delivery of a nucleic acid molecule to a cell in an animal.

Preferably, the animal is a human.

Preferably, the copolymers according to the present invention are substantially non-toxic to animal cells at concentrations typically used in gene therapeutic applications. Typical concentrations are in the order of about 25 to 6000 μM, preferably about 60 to 4000 μM can be used for in vitro and in vivo applications without significant toxicity to cells. It will be appreciated that the concentration required will depend on the actual copolymer used, the amount, type or form of nucleic acid to be delivered, and the target cell type.

Preferably, the copolymer has a net positive charge and is capable of containing or complexing with negatively charged nucleic acid molecules such as DNA and RNA. The net positive is provided by one or more of primary, secondary and tertiary amines that may interact with negatively charged nucleic acid molecules.

By low molecular weight polyethylenimine, it is meant to include polyethylenimine having a molecular weight of less than about 25000. The polyethylenimine monomers of choice typically have a molecular weight of less than about 20000, preferably less than about 10000, more preferably less than about 5000. The present inventors have found that copolymers formed from polyethylenimine having molecular weights from about 500 to 4000, preferably between about 600 to 2000, are particularly suitable for delivering nucleic acid molecules to cells according to the present invention.

Preferably, the cyclodextrin is a modified cyclodextrin. More preferably, the cyclodextrin is β-cyclodextrin modified or activated to allow attachment to polyethylenimine. In a preferred form, the β-cyclodextrin is activated by β-1,1′-carbonyldiimidazole. Although β-1,1′-carbonyldiimidazole has been found to be suitable to activate cyclodextrin, other agents suitable for this purpose include but are not limited to benzotriazole carbonate, N,N′-disuccinimidyl carbonate, chloroformates, N-hydroxysuccinimidyl chloroformate, and carbonylimidazole.

The linking of the polyethylenimine through the cyclodextrins is usually by carbonyl group chemical linkage.

Preferably, the copolymer contains ester bonds that can be hydrolyzed under physiological conditions.

The copolymer can contain up to about 35 units of polyethylenimine linked by cyclodextrins. Preferably, the copolymer contains between about 5 and 25 polyethylenimine units, more preferably about 10 to 15 polyethylenimine units linked by cyclodextrins.

The biodegradable copolymer is capable of forming a complex with negatively charged nucleic acids, including DNA, RNA and oligonucleotides. Preferably, the nucleic acid is in the form of a plasmid containing nucleic acid molecule having a nucleotide sequence which, when internalized in a cell, can provide some desired beneficial effect to the cell.

The biodegradable copolymers can be diluted or dispersed in any suitable medium for application to cells. Similarly, the copolymers can be formulated in pharmaceutically acceptable diluents for administration to animals including humans. Such formulation is well known to the art.

The copolymers can be administered to animals by any suitable route. Typically, the copolymers containing the nucleic acid molecules are administered by injection, typically at the desired site.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

In order that the present invention may be more clearly understood, preferred forms will be described with reference to the following drawings and examples.

### BRIEF DESCRIPTION OF THE DRAWINGS

FIG. 1A shows a synthesis scheme of PEI-CyD copolymers according to the present invention.

FIG. 1B shows chemical linkage of PEI-CyD copolymers according to the present invention.

FIG. 2 shows the ¹H NMR spectra of 1,1′-carbonyldiimidazole (CDI) in D₂O, CyD in DMSO and in CyD in D₂O.

FIG. 3 shows the ¹H NMR spectra of β-CyD-CDI at various concentrations.

FIG. 4 shows CyD/PEI molar ratios calculated according to the integral of proton NMR spectrum.

FIG. 5 shows mass spectral analysis of PEI2000-CyD.

FIG. 6 shows GPC analysis of PEI600-CyD (upper) and PEI1200-CyD (lower).

FIG. 7 shows gel retardation assays of various PEI-CyD copolymers complexed with DNA.

FIG. 8 shows gel retardation assays of PEI600-CyD/DNA complex.

FIG. 9 shows results of cell viability assays of various PEI-CyD copolymers.

FIG. 10 shows results of transfection efficiencies of various cross-linked copolymers in seven different cell lines using a luciferase reporter gene under a CAG promoter.

FIG. 11 shows results of transfection efficiencies of the various cross-linked products in seven different cell lines using a luciferase reporter gene under a CAG promoter.

FIG. 12 shows release of DNA from copolymer complex using an agarose gel assay.

FIG. 13 shows results of copolymer degradation as measured by viscosity analysis.

FIG. 14 shows results of DNA release from copolymers in an in vivo study using a rat spinal cord assay.

### MODE(S) FOR CARRYING OUT THE INVENTION

DEFINITIONS

| | |
|---|---|
| PEI | polyethylenimine |
| PEI600 | polyethylenimine having a molecular weight of 600 |
| PEI1200 | polyethylenimine having a molecular weight of 1200 |
| PEI2000 | polyethylenimine having a molecular weight of 2000 |
| PEI25k | polyethylenimine having a molecular weight of 25,0000 |
| CyD | cyclodextrin |
| β-CyD | β-cyclodextrin |
| PEI-CyD | polyethylenimine/cyclodextrin copolymer |
| PEI600-CyD | polyethylenimine 600/cyclodextrin copolymer |
| PEI1200-CyD | polyethylenimine 1200/cyclodextrin copolymer |
| PEI2000-CyD | polyethylenimine 2000/cyclodextrin copolymer |
| CDI | 1,1′-carbonyldiimidazole |
| CDI-CyD | β-cyclodextrin-carbonate-benzotriazole |

Materials & Methods

Polymer Synthesis

Polyethylenimine (PEI), average molecular weight 600, 1200, and 2000, β-cyclodextrin (CyD, molecular weight 1,135) and 1,1′-carbonyldiimidazole (CDI) were used for copolymer synthesis. 0.42 g β-cyclodextrin (0.00037 mol) and 0.80 g 1,1′-carbonyldiimidazole (0.0052 mol) were dissolved in 6 ml N,N-dimethylformamide (DMF). The mixture was stirred at room temperature and reacted for 1 h. Reaction system was protected by nitrogen. The mixture was precipitated in cold ethyl ether, filtered, dissolved in 5 ml dimethlsulfoxide DMSO, stored at 4° C. PEI polymers (1.5 g, 0.0025 mol) were dissolved in 3 ml DMSO. After PEI dissolved in the reaction solution, CyD-CDI in 5 ml DMSO and 0.3 ml triethylamine (Et3N) was added drop wise over 1.5 h. After additional stirring for 4-5 h, the mixture was dialyzed in water and freeze-dried for 2 days.

Polymer Characterization

¹H NMR Analysis

The structures of PEI600-CyD and PEI2000-CyD were ascertained by ¹H NMR. The ratio of cyclodextrin and polyethylenimine in the copolymer sample was determined from ¹H NMR spectra using integral values obtained for the —H protons of cyclodextrin rings and —CH₂CH₂NH— protons of PEI. The ¹H NMR analysis was carried out with 20 mg of PEI-CyD sample dissolved into 0.7 ml deuterium oxide (D₂O) in Bruker 400 MHz NMR spectrometer with 32 scans at room temperature.

Mass Spectrum Analysis

PEI2000-CyD was analyzed by MALDI-TOF mass spectrometry (MALDI-TOF-MS) (Voyager DESTR Perkin-Elmer PerSeptive Biosystem) operated in the liner mode. The matrix for PEI2000-CyD was α-cyano-4-hydroxycinnamic acid. A 337 nm N₂ laser was used with at least 200 shots on average for the final spectrum.

Gel Perforation Chromatography (GPC) Analysis

Molecular weights of synthesized copolymers were determined by GPC using Waters 600E pump and Waters 410 Refractive Index Detector (33° C.). The columns were Phenomenex Polysep Guard S/n 70978G, Polysep GFC-P S/n 70977 and Polysep GFC-P S/n 70976 (33° C.). PEI600-CD and PEI2000-CD samples were run at a 10 mg/ml concentration in distilled-water. The running solution used was distilled water, with a flow rate of 0.8 ml/min. The injected volume was 300 μl. Molecular weight analyses were made against five poly(ethylene glycol) standard of number average molecular weights 7100, 10600, 12600, 23600 and 56000.

Agarose Gel Electrophoresis

Agarose electrophoresis (1% agarose containing ethidium bromide (EtBr)) was used to determine copolymers and DNA binding. Varying amounts of PEI600-CD and PEI2000-CD were mixed with 0.1 μg of DNA in 5% glucose solution and loaded into the gel. Gel electrophoresis was run at room temperature in TEB buffer at 80 V for 60 min. DNA bands were visualized by a UV illuminator and electrophoretic mobility of DNA samples complexed with the copolymers was measured.

Particle Size of Copolymer-DNA Complexes

Complexes were prepared at a DNA concentration of 0.2 mg/ml, N/P ratio=20 for PEI2000, PEI2000-CyD, PEI600 and PEI600-CyD. A volume of 1 ml was used for the measurement. Sizes were measured with N4 Plus Submicron Particle Sizer (COULTER, USA) at room temperature. Scattering light was detected at 90° angle, running of 200 sec for each sample and analyzed in the Unimodal Analysis mode. For data analysis, the refractive index medium (1.332) of 5% glucose at 20° C. was detected.

Cytotoxicity Assay

Cos7, NT2, MCT17.2, U256, 293, KB3-1 and HEPG2 cells were used. Cells were cultured in DMEM supplemented with 10% FCS at 37° C., 10% CO₂, and 95% relative humidity. C17.2 cells were cultured in Dulbecco's modification of Eagle's minimum essential medium (DMEM) supplemented with 10% fetal calf serum (FCS) and 5% heat-inactivated horse serum. For cell viability assay, copolymer solutions were prepared in serum supplemented tissue culture medium. pH and osmolarity of the preparations were routinely measured and adjusted to pH 7.4 and 280-320 mosm/kg. The cells (10,000 cells/well) were seeded into 96-well microtiter plates (Nunc, Wiesbaden, Germany). After overnight incubation, the culture medium was replaced with 100 μl serial dilutions of copolymers to be tested and the cells were incubated for another 24 h. Twenty μl sterile filtered MTT (3-(4,5-dimethyl-thiazol-2-yl)-2,5-diphenyl tetrazolium bromide) (5 mg/ml) stock solution in phosphate buffered saline (PBS) were added to each well reaching a final concentration of 0.5 mg MTT/ml. After 4 h un-reacted dye was removed by aspiration. The formazan crystals were dissolved in 100 μl/well DMSO (BDH laboratory Supplies, England) and measured spectrophotometrically in an ELISA reader (Model 550, Bio-Rad) at a wavelength of 655 nm. The spectrophotometer was calibrated to 0 absorbance using culture medium without cells. The relative cell growth (%) related to control cells containing cell culture medium without copolymer was calculated by [A] test/[A] control×100.

Gene Transfection Assay

For in vitro transfection studies, cells were seeded 24 h prior to transfection into a 24-well plate (Becton-Dickinson, Lincoln Park, N.J.) at a density of 5×10⁴ per well with 0.5 ml of indicated medium. The plasmid used was pCAGLuc, encoding firefly luciferase driven by a composite promoter CAG consisting of the CMV IE enhancer, chicken β-actin promoter and rabbit β-globin polyadenylation (poly-A) signal. At the time of transfection, the medium in each well was replaced with 300 μl of Opti-MEM. PEI-DNA or PEI-CyD-DNA complexes were incubated with the cells for 3 h at 37° C. The medium was then replaced with 0.5 ml of fresh complete medium and cells were further incubated for 24 h. After incubation, cells were permeabilized with 100 μl of cell-lysis buffer (Promega Co, Wis USA). The luciferase activity in cell extracts was measured using a luciferase assay Kit (Promega Co., Madison, Wis. USA) on a single-well luminometer (Berthold Lumat LB 9507, Germany) for 10 s. The light units (LU) were normalized against protein concentration in the cell extracts, which was measured using a protein assay kit (Bio-Rad Labs, Hercules, Calif.).

In Vivo Gene Transfection Assay

The plasmid used was pCAG-luc, encoding firefly luciferase driven by a composite promoter CAG consisting of the CMV IE enhancer, chicken β-actin promoter and rabbit β-globin polyadenylation signal. Complexes were prepared by mixing DNA and copolymers in 5% glucose solution. All solutions used for in vivo transfection experiments were prepared in distilled water containing 5% glucose. The mixture with PEI25k (N/P ratio was 15/1) was prepared as follows: 4.5 μl of PEI25k (0.1M) in 45.5 μl 5% glucose was added to 12.5 μg of pCAG-luc in 50 μl 5% glucose solution, vortexing for 1 min. The mixture with PEI600-CyD (N/P ratio was 20/1) was prepared as follows: 6.0 μl of PEI600-β-CyD (0.1M) in 44.0 μl 5% glucose was added to 12.5 μg of pCAG-luc in 50 μl 5% glucose solution, vortexing for 1 min. The mixture with PEI600 (N/P ratio was 20/1) was prepared as follows: 6.0 μl of PEI600 (0.1M) in 44.0 μl 5% glucose was added to 12.5 μg of pCAG-luc in 50 μl 5% glucose solution, vortexing for 1 min. After mixing, the transfection systems were allowed to incubate at room temperature for 30 min.

Female Swiss albino mice, 20-25 grams, obtained from NUS Laboratory Animal Center were used. Mice were briefly anesthetized by inhalation of ether in air and placed in a prone position with their neck draped over plastic tube having a diameter of approximately 10 mm. The head was immobilized with the thumb and midfinger and the index finger palpating the space between the occiput and the first cervical vertebra. The present inventors intracisternally injected 10 mice for each group and 10 μl of the prepared nano-particles with Hamilton syringe with a stop at 4 mm of the tip of the needle to each mouse. For sample analysis, the injected mice were kept in air-conditioned room, free feeding and drinking, 12 h daylight and night cycle for 24 h. The mice were sacrificed after perfusion via left ventricle with PBS under anesthetic with ether and the cerebella, cerebrum and brain stem were collected together for measuring the activity of luciferase.

Results

Copolymer Characterization

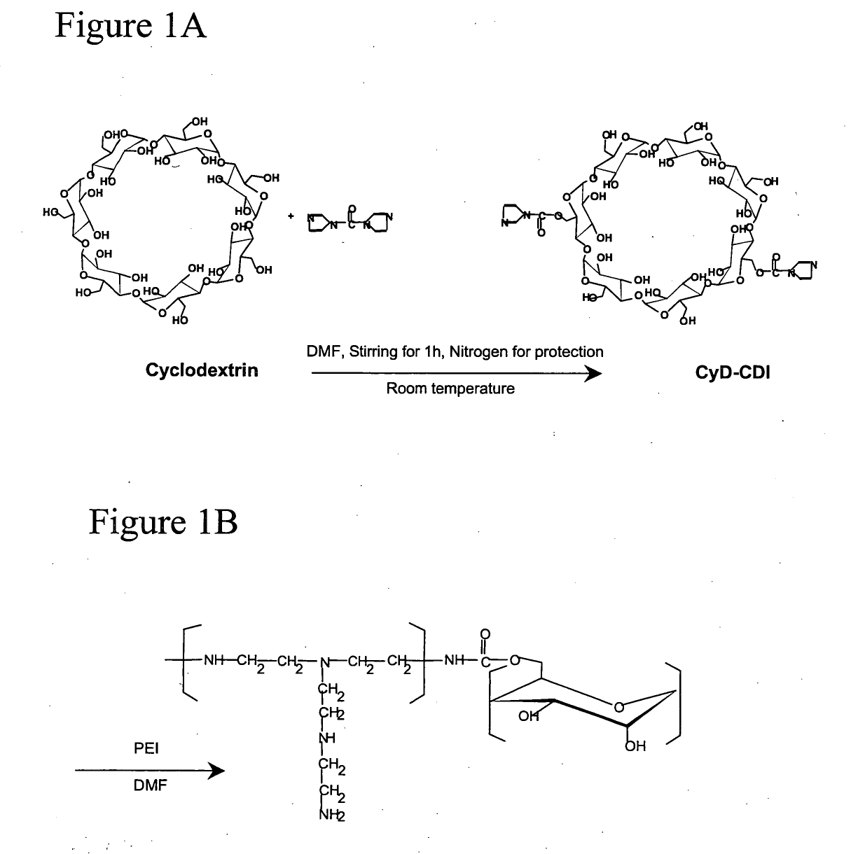

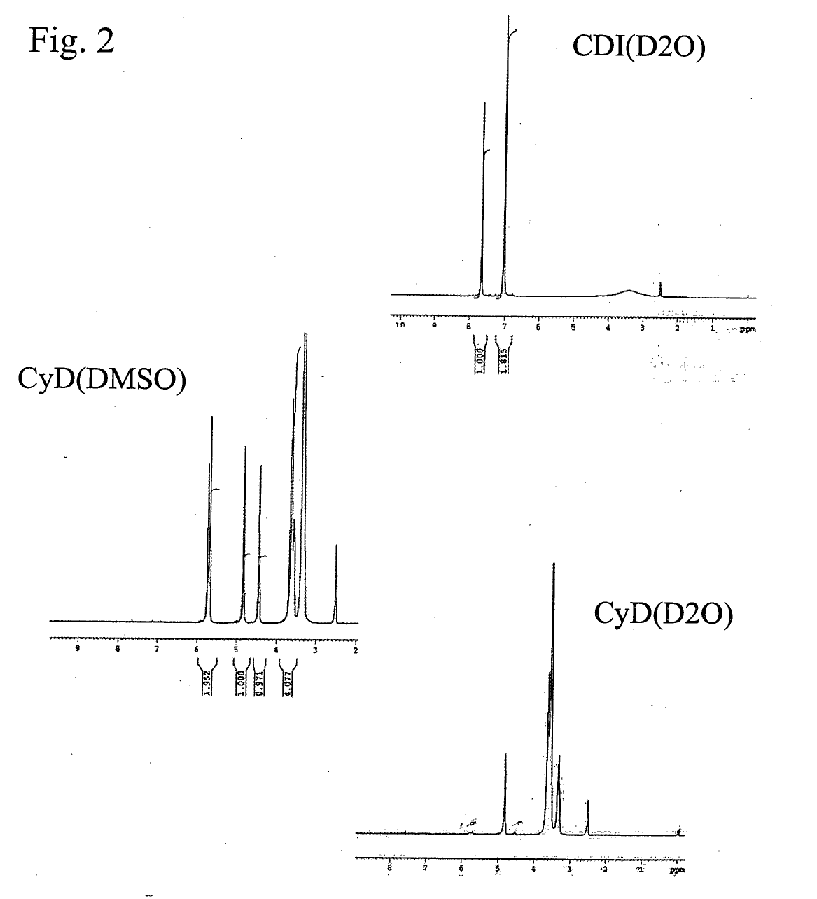

FIG. 1A shows the synthesis scheme of PEI-CyD copolymers and FIG. 1B indicates the chemical linkage in more detail of PEI-CyD copolymers according to the present invention. The synthesis procedure involved the preparation of the activated β-cyclodextrin (β-cyclodextrin-carbonate-benzotriazole, CDI-CyD) and synthesis of β-cyclodextrin-polyethylenimine (PEI-β-CyD). The ¹H NMR spectra of 1,1′-carbonyldiimidazole (CDI) in D₂O, CyD in DMSO and in D₂O are shown in FIG. 2. In the solvent of DMSO, all protons appeared in the spectrum, but D₂O solvent changed the active hydroxyl groups. It was clear that the peaks at δ 5.6-5.8 ppm should be assigned to hydroxyl groups inside ring of β-cyclodextrin, and δ 4.5 ppm was for hydroxyl groups outside ring of β-cyclodextrin. Compared with the standard spectrum of β-cyclodextrin, the peak at δ 4.8 ppm was assigned to the number 1 hydroxyl of β-cyclodextrin.

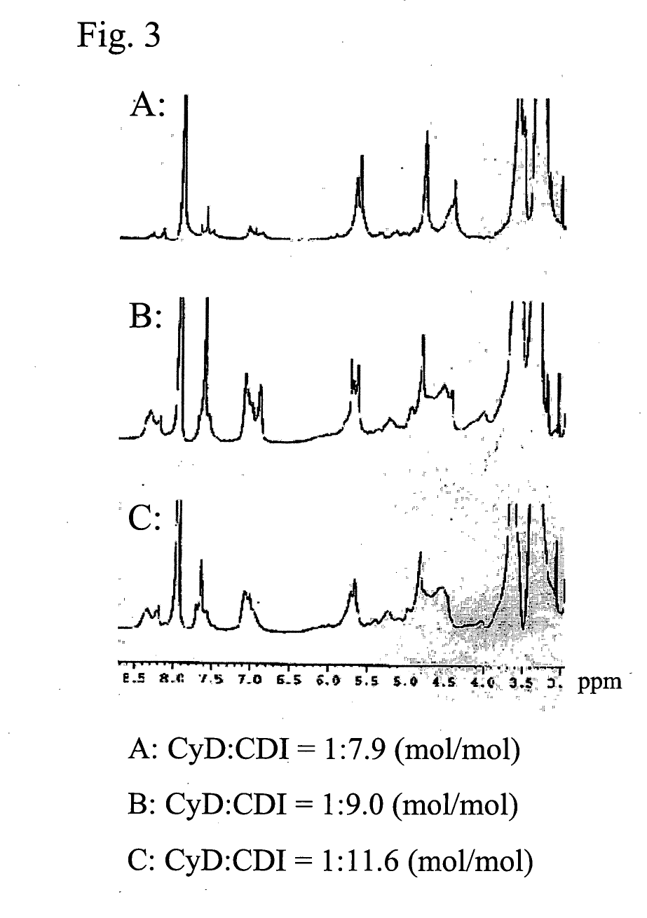

FIG. 3 shows the ¹H NMR spectra of β-CyD-CDI. Changing the amount of materials used, CDI-linked CyD with various ratios can be prepared. With the increase in the amount of CDI, the peak at 4.5 ppm was decreased, indicating that the protons from C6 hydroxyl group of the β-cyclodextrin ring were decreased due to reaction with CDI. The ratios were determined from ¹H NMR by comparing the integral values obtained from the numbers of the H proton of β-CyD in C1 and OH proton of β-CyD in C6. Three ratios, 15, 22 and 33% of hydroxyl group could be achieved. For a linear structure copolymer to be obtained, the β-CyD with modification degree of 33% was used for the reaction with PEI.

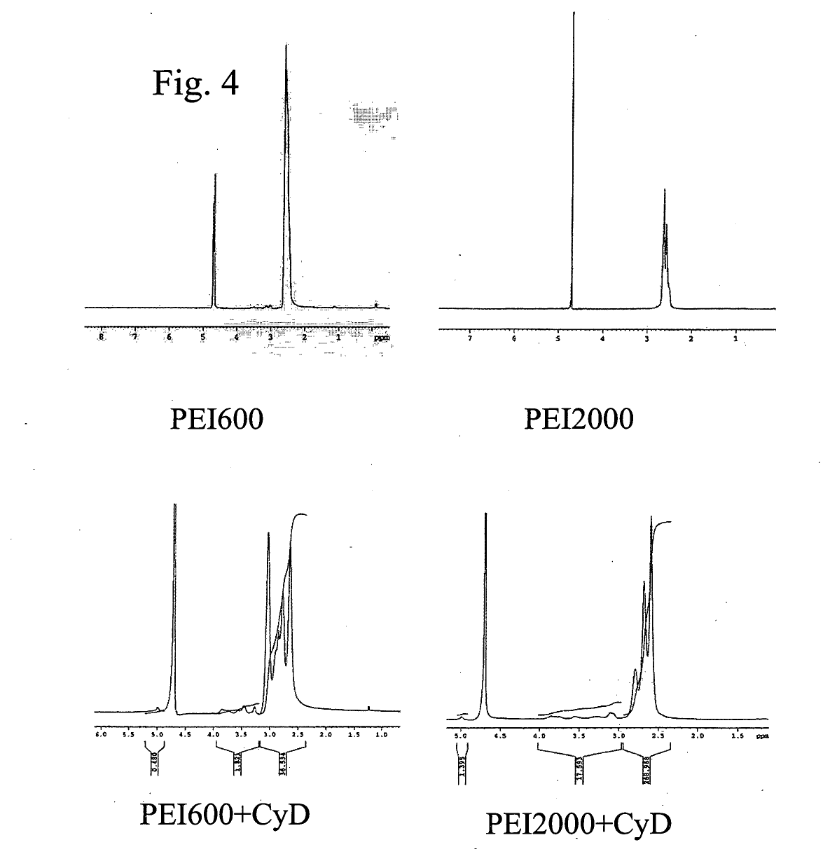

After the reaction, CyD/PEI molar ratios were calculated according to the integral of proton NMR spectrum (FIG. 4). The formula of calculation was as follows:

CyD/PEI=(A/1×7)/(B/4×46.5)

where A: integral of 1H in glucose unite of β-cyclodextrin; and

B: integral of proton in PEI (CH₂CH₂NH) unit.

Newly synthesized PEI2000 and PEI600 copolymers contained 46.5 and 14 units, respectively.

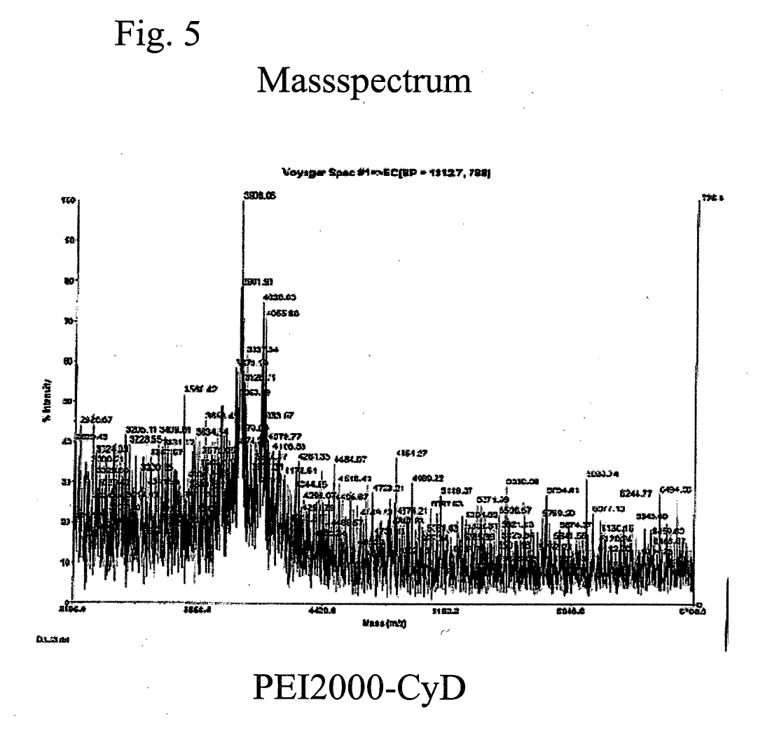

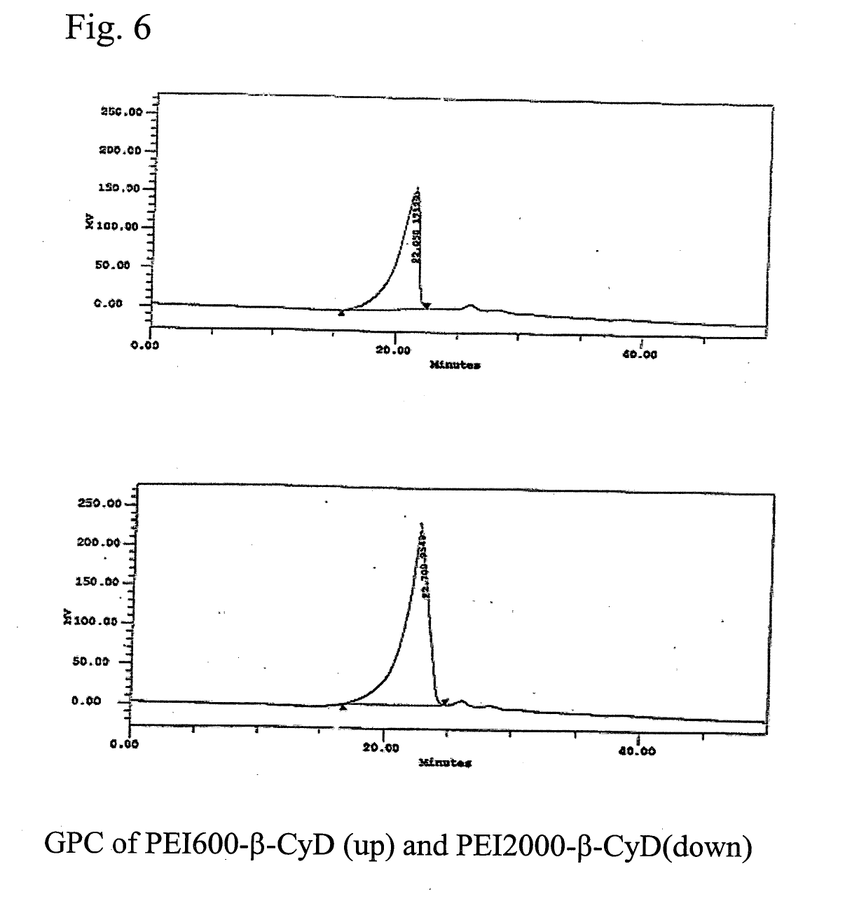

Mass spectral analysis of PEI2000-CyD showed dominant peaks at MW 3900 to 4000 and confirmed the linkage of PEI2000 and cyclodextrin (FIG. 5). The GPC analysis indicated that the molecular weights, increased from 600 to 63,000 and 2000 to 19,000, respectively (FIG. 6), confirming the cross-linking of the low molecular weight PEIs by CyD. In one preparation, the molecular weight of PEI2000-CyD reached 40,000.

The PEI-CyD copolymers were able to complex DNA to form particles. Size measurement with light scattering indicated particles formed using PEI-CyD were in the range of 90 to 130 nm, smaller than those formed using PEI polymers (see Table). More importantly, these PEI-CyD/DNA particles had a much narrow distribution of sizes. Most of the copolymers produced, 80 to 90%, had sizes around 90 or 130 nm, depending on which PEI was used.

TABLE
Particle sizes of polymer/DNA complexes*

| Polymer | n | Mean | SD | % |
|---|---|---|---|---|
| PEI2000 | 6 | 163 | 25 | 66.4 |
| PEI2000-CyD | 8 | 130.4 | 43.5 | 97 |
| PEI600 | 6 | 94.2 | 16.2 | 45.5 |
| PEI600-CyD | 7 | 87.6 | 21.2 | 82.4 |

*The particles were prepared at a concentration of DNA 0.1 μg/μl and N/P 20/1.

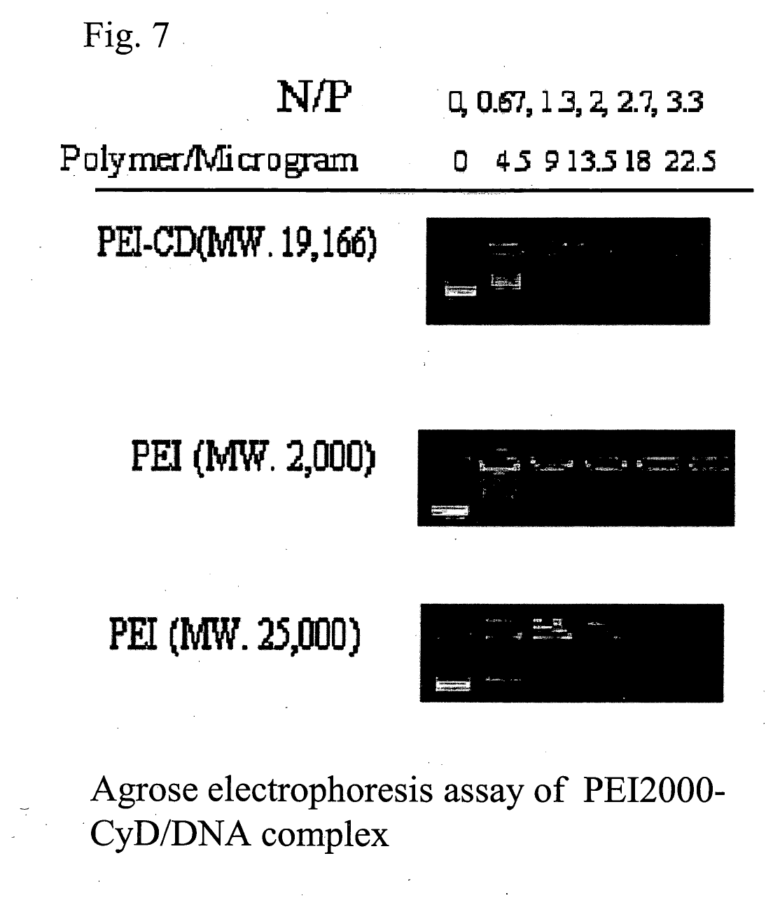

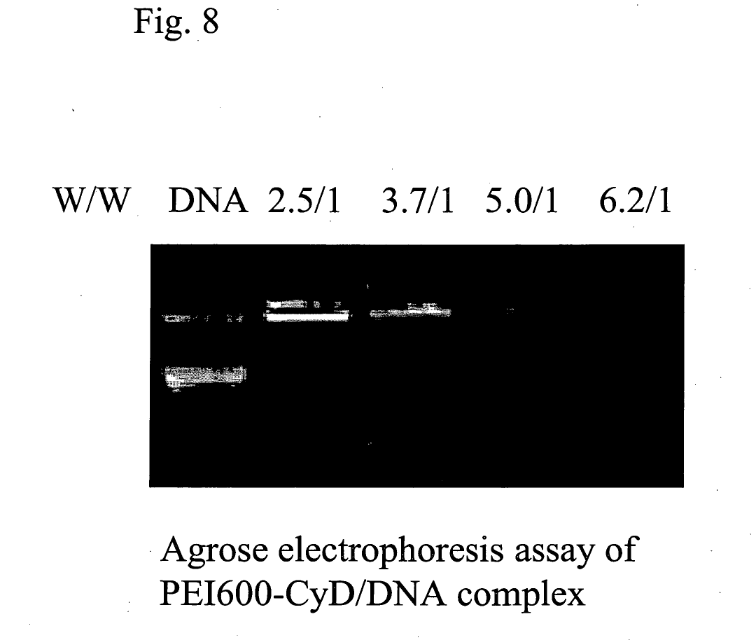

The PEI-CyD copolymers were capable of binding, neutralizing and condensing DNA. As showed in gel retardation assays in FIG. 7 and FIG. 8, with the increase in the amount of the copolymers used, charges of DNA were neutralized and DNA migration in agarose gel slowed down. Higher amounts of the copolymer stopped the migration totally.

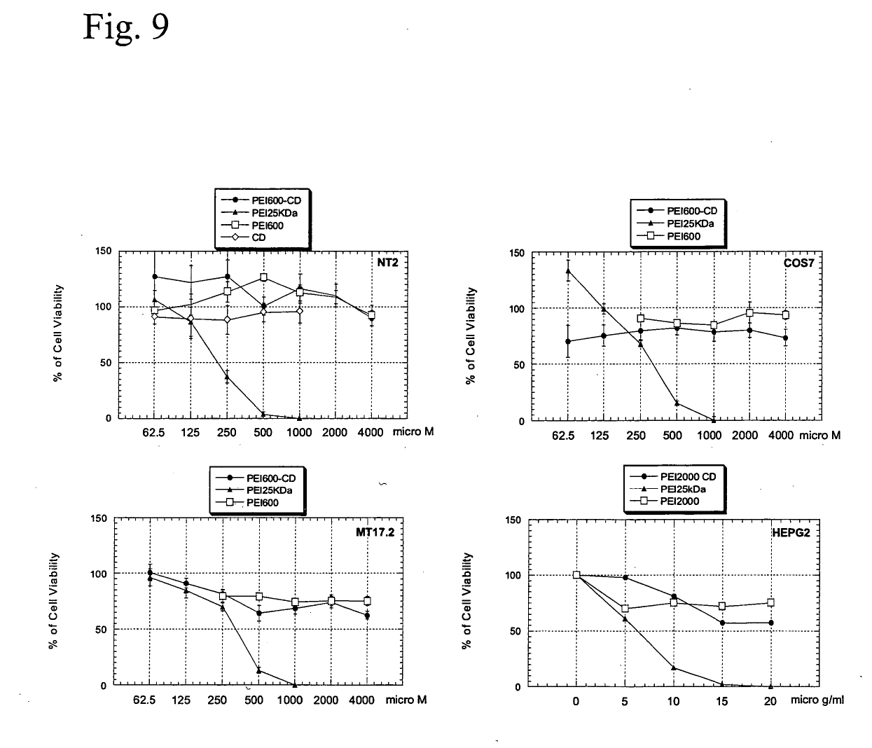

The cell viability assay indicated that PEI600-CyD and PEI2000-CyD exhibited much lower cytotoxicity in cultured cells than PEI25k (FIG. 9). At a concentration of 1 mM, cell viability was 80% in COS7, 100% in NT2 cells, and over 70% in C17.2 cells when PEI600-CyD, as well as PEI600, were used. Whereas all cells died in the PEI25k test group. Similar results were observed in PEI2000-CyD group. A dose of 15 μg/ml caused 100% of death in HEPG2 cells, 50% of cells survived in PEI-CyD group. When compared with monomer CD, PEI600-CyD showed a relatively lower cytotoxicity.

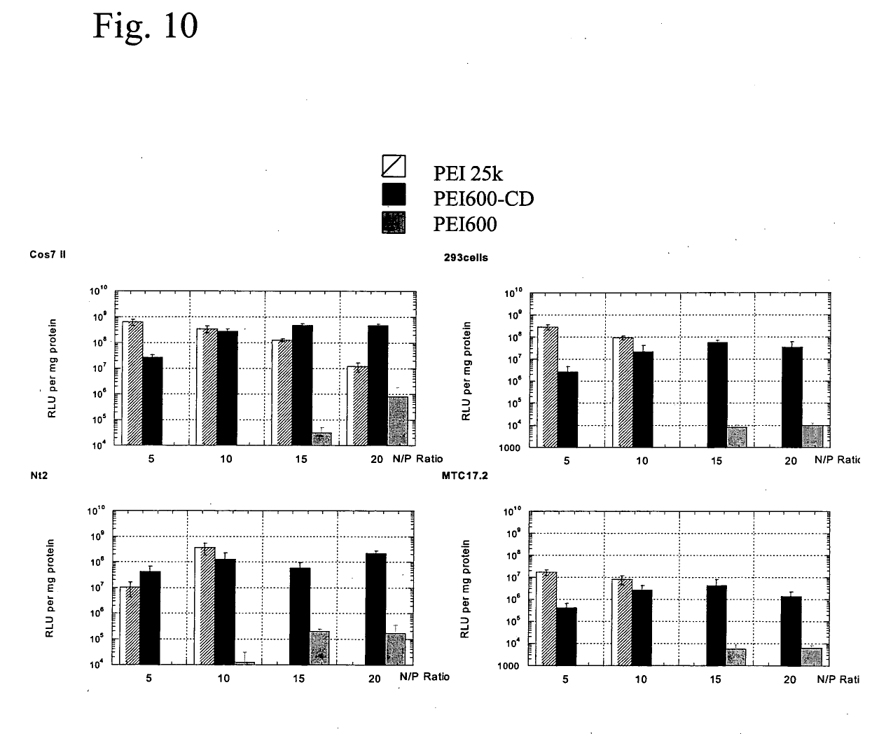

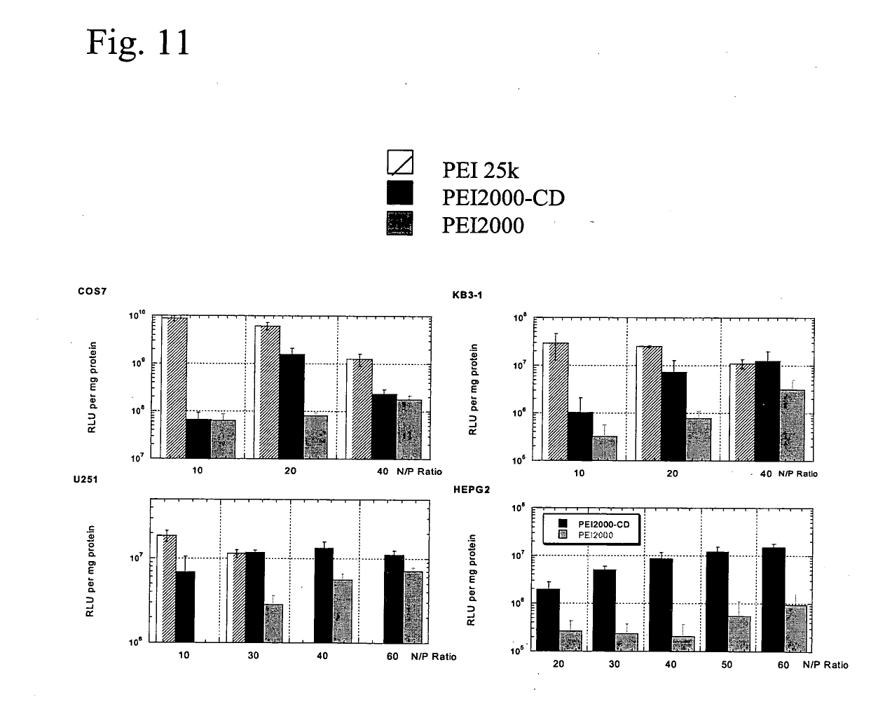

The transfection efficiencies of the resulting cross-linked products were evaluated in seven different cell lines using a luciferase reporter gene under a CAG promoter. The results showed that cross-linked copolymers mediated variable levels of transfection, depending on the cross-linking reagent, the extent of conjugation, and the N/P ratio. As shown in FIG. 10 and FIG. 11, the luciferase expression levels increased when PEI600-CyD and PEI2000-CyD were used instead of the low MW PEI polymers for in vitro cell gene transfection. At N/P ratio 40, the transfection efficiency was about 1000 times higher in NT2 cells, 100 times in COS7 cells and 100 times in C17.2 cells. In addition, in vitro transfection efficiencies with the PEI-CyD copolymers were comparable to the best results obtained with PEI25k.

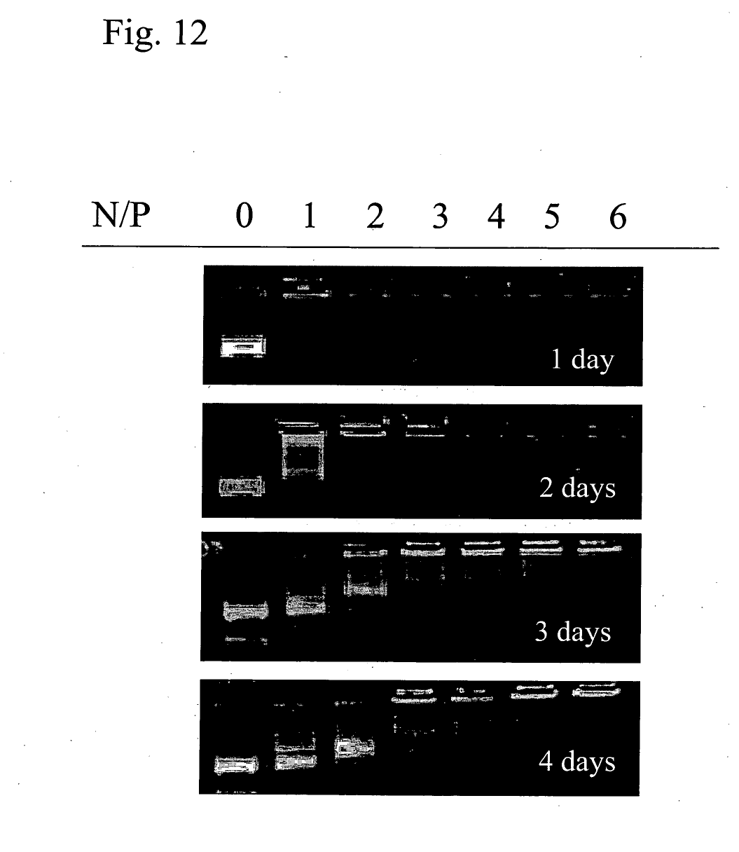

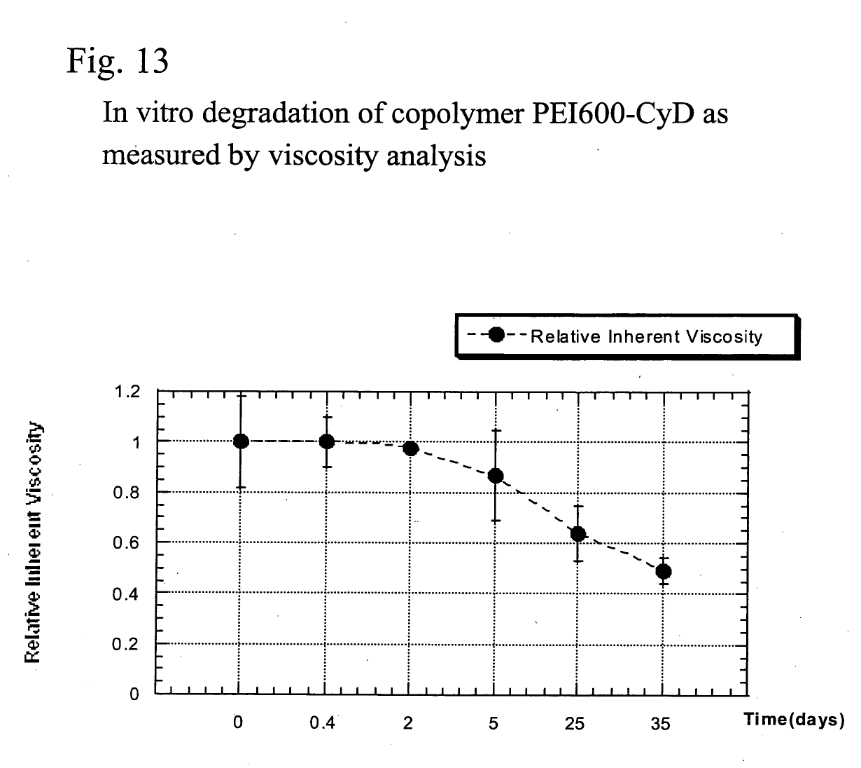

PEI-CyD copolymers developed according to the present invention undergo hydrolytic degradation in PBS (pH 7.4) at 37° C., because of ester bonds formed. Within 25 days, the molecular weight of the copolymer decreased from 63000 to 30000. With the degradation, plasmid DNA was released from copolymer complexes. The release rate of DNA was a function of charge ratio. Complexes at the N/P ratio of 1 started to release DNA after 2 days of incubation in PBS at 37° C. as indicated by agarose gel analysis (FIG. 12). Higher charge ratios lead to slower release of DNA from the complexes. At the N/P ratio of 6, DNA release started to appear at day 3. The electrophoretic mobility of DNA release from the complexes was slightly lower, probably because of the effects of the degraded low molecular weight PEI600 oligomers. As well, the viscosity analysis of the copolymer dissolved in PBS showed a 60% decrease within 36 days (FIG. 13).

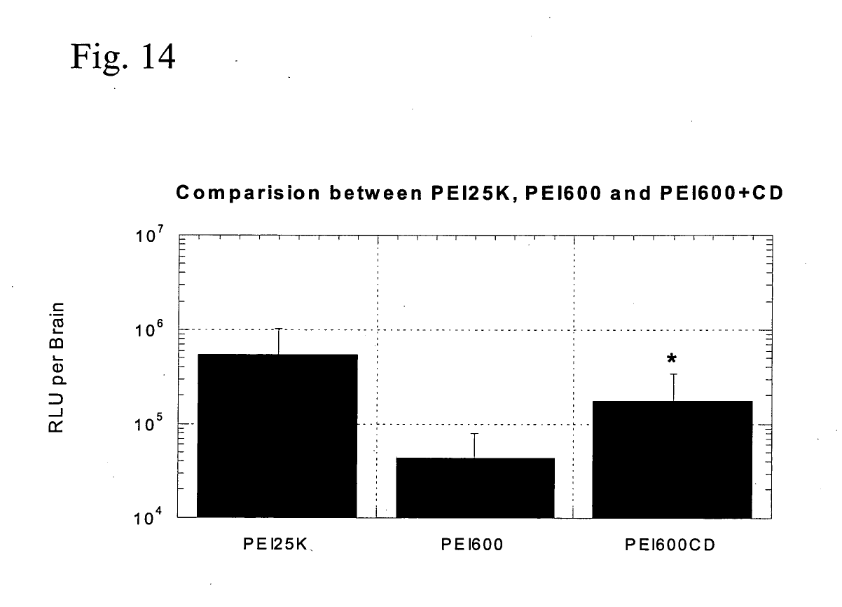

An in vivo study was carried out in the rat spinal cord through intrathecal administration of plasmid DNA complexed with CyD-PEI into the cerebrospinal fluid. These complexes provided transgene expression in the spinal cord 10 to 20-fold higher than naked plasmid DNA. Compared with PEI600, PEI600-CyD enhanced the delivery of plasmid DNA into the CNS significantly (P<0.05, FIG. 14), with a 4-fold higher level of gene expression. Even though the transfection efficiency mediated by PEI600-CyD was not as high as that of PEI25k in this in vivo study, much lower cytotoxicity of CyD-PEI600 was demonstrated in the in vitro trials. These results suggest the possibility for safer delivery of genetic materials, especially when a repetitive administration scheme is required. Thus, this new class of cross-linked copolymers provides a viable non-viral, chemical-based DNA delivery system for gene therapy.

Summary

The present invention provides biodegradable copolymers, synthesized through low molecular weight polyethylenimine (typically less than about 2,000) cross-linked by modified β-cyclodextrin. The terminal β-cyclodextrin hydroxyl group was activated by 1,1′-carbonyldiimidazole. Each activated β-cyclodextrin links two or three units of low molecular weight PEI to form a linear structure copolymer.

Modification degree (%) of β-cyclodextrin and 1,1′-carbonyldiimidazole were calculated by proton NMR, showing 33% of hydroxyl group in β-cyclodextrin being modified. Activated β-cyclodextrin was reacted with low molecular weight PEI to form linear structure of PEI-CyD. Ester bonds were formed during the reaction of activated CyD and PEI. GPC measurement showed that the molecular weight of the synthesized copolymers increased from 600 to 23,000 and 2,000 to 19,000 for PEI600-CyD and PEI2000-CyD, respectively. Typically the copolymers of the invention contain 10 or 15 PEI-CyD units.

The developed products are degradable in the body and displayed much less toxicity and almost same transfection efficiency as PEI25k in cultured cells.

The present invention offers a new biodegradable PEI-CyD copolymer that can bind and condense DNA and mediate intracellular gene transfection. Low molecular weight PEI were linked with β-cyclodextrin that had been modified by 1,1′-carbonyldiimidazole to form a linear structure copolymer. Some features of the copolymers according to the present invention include:

I. PEI-CyD copolymers of the invention, being different from non-degradable PEI25k, are degradable in PBS buffer solution or in physiological condition, because of ester bonds formed during CyD-PEI linkage.

II. PEI-CyD copolymers of the invention are biocompatible materials, showing no toxicity to all cells tested. This is strikingly different from PEI25k, which causes cell death at relatively lower doses. The degradation products of the copolymers are low MW PEI and CyD, which are also not toxic.

III. PEI-CyD copolymers of the invention typically have a net positive charge. Positively chargeable groups include primary, secondary and tertiary amines. The copolymers may efficiently bind and condense plasmid DNA. The copolymers are probably able to disrupt endosome membranes once endocytosed into cells and promote the endosome escape or release of DNA molecules. The copolymers mediate gene expression efficiently in both cells and animals. The transfection efficiency of the copolymers is much higher than those mediated by a low MW PEI or CyD alone and close to that offered by PEI25k.

The present inventors have used cyclodextrins to cross-link low molecular weight PEI polymers to increase the number of amine groups per each copolymer molecule, thus enhancing the capability of the PEI polymers in binding, neutralizing and condensing DNA, yet retaining their low toxicity. Biodegradable ester bonds can also be introduced during the activation of CyDs and the conjugation of activated CyD with PEI. By combining PEI and CyD molecules together, the resulting copolymers have been surprisingly found to act in a synergistic manner as a safe and effective delivery system for nucleic acids.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A biodegradable copolymer suitable for delivering a nucleic acid molecule to a cell, the copolymer comprising low molecular weight polyethylenimine cross-linked by a cyclodextrin.

2. The biodegradable copolymer according to claim 1 having a net positive charge and being capable of complexing with negatively charged nucleic acid molecules.

3. The biodegradable copolymer according to claim 2 wherein the net positive charge is provided by one or more of primary, secondary and tertiary amines.

4. The biodegradable copolymer according to any one of claims 1 to 3 wherein the low molecular weight polyethylenimine has a molecular weight of less than about 25000.

5. The biodegradable copolymer according to claim 4 wherein the polyethylenimine has a molecular weight of less than about 20000, preferably less than about 15000, more preferably less than about 10000, even more preferably less than about 5000.

6. The biodegradable copolymer according to claim 4 wherein the polyethylenimine has a molecular weight of less than about 2000.

7. The biodegradable copolymer according to claim 6 wherein the polyethylenimine has a molecular weight from about 600 to 2000.

8. The biodegradable copolymer according to any one of claims 1 to 7 wherein the cyclodextrin is β-cyclodextrin modified or activated by an agent to allow attachment to polyethylenimine.

9. The biodegradable copolymer according to claim 8 wherein the agent is selected from the group consisting of β-1,1′-carbonyldiimidazole, benzotriazole carbonate, N,N′-disuccinimidyl carbonate, chloroformates, N-hydroxysuccinimidyl chloroformate, and carbonylimidazole.

10. The biodegradable copolymer according to claim 9 wherein the agent is β-1,1′-carbonyldiimidazole.

11. The biodegradable copolymer according to any one of claims 1 to 10 wherein the polyethylenimine is cross-linked to cyclodextrin via a carbonyl group.

12. The biodegradable copolymer according to claim 11 having ester bonding.

13. The biodegradable copolymer according to any one of claims 1 to 12 wherein the copolymer contains up to about 35 polyethylenimine units.

14. The biodegradable copolymer according to claim 13 wherein the copolymer contains between about 5 and 25 polyethylenimine units.

15. The biodegradable copolymer according to claim 14 wherein the copolymer contains about 10 to 15 polyethylenimine units.

16. A method for synthesizing a biodegradable copolymer comprising the steps of:
(a) treating cyclodextrin with an agent to form a modified or activated cyclodextrin; and
(b) adding the modified or activated cyclodextrin to a low molecular weight polyethylenimine to form a mixture and treating the mixture under suitable conditions to form a biodegradable copolymer comprising polyethylenimine linked by cyclodextrin.

17. The method according to claim 16 wherein the cyclodextrin is β-cyclodextrin.

18. The method according to claim 17 wherein the agent is selected from the group consisting of β-1,1′-carbonyldiimidazole, benzotriazole carbonate, N,N′-disuccinimidyl carbonate, chloroformates, N-hydroxysuccinimidyl chloroformate, and carbonylimidazole.

19. The method according to claim 16 wherein the agent is β-1,1′-carbonyldiimidazole.

20. The method according to any one of claims 16 to 19 wherein the low molecular weight polyethylenimine has a molecular weight of less than about 25000.

21. The method according to claim 20 wherein the polyethylenimine has a molecular weight of less than about 20000, preferably less than about 15000, more preferably less than about 10000, even more preferably less than about 5000.

22. The method according to claim 20 wherein the polyethylenimine has a molecular weight of less than about 2000.

23. The method according to claim 22 wherein the polyethylenimine has a molecular weight from about 600 to 2000.

24. The method according to any one of claims 16 to 23 wherein the polyethylenimine is cross-linked to cyclodextrin by a carbonyl group.

25. The method according to claim 24 wherein the copolymer contains ester bonding.

26. The method according to any one of claims 16 to 25 wherein the copolymer contains up to about 35 polyethylenimine units.

27. The method according to claim 26 wherein the copolymer contains between about 5 and 25 polyethylenimine units.

28. The method according to claim 27 wherein the copolymer contains about 10 to 15 polyethylenimine units.

29. A biodegradable copolymer synthesized by the method according to any one of claims 16 to 28.

30. A method for delivering a nucleic acid molecule to a cell, the method comprising forming a complex between a biodegradable copolymer according to any one of claims 1 to 15 and a nucleic acid molecule and exposing the cell to the copolymer/nucleic acid molecule complex such that the complex is internalized by the cell and the nucleic acid molecule is released in the cell.

31. The method according to claim 30 wherein the cell is in an animal.

32. The method according to claim 31 wherein the animal is a human.

33. A method for delivering a nucleic acid molecule to a cell, the method comprising forming a complex between a biodegradable copolymer according to claim 29 and a nucleic acid molecule and exposing the cell to the copolymer/nucleic acid molecule complex such that the complex is internalized by the cell and the nucleic acid molecule is released in the cell.

34. The method according to claim 33 wherein the cell is in an animal.

35. The method according to claim 34 wherein the animal is a human.

36. Use of a biodegradable copolymer according to any one of claims 1 to 15 to deliver a nucleic acid molecule to a cell.

37. Use of a biodegradable copolymer according to claim 29 to deliver a nucleic acid molecule to a cell.

38. Use of a biodegradable copolymer according to any one of claims 1 to 15 in the manufacture of a medicament for the delivery of a nucleic acid molecule to a cell in an animal.

39. Use of a biodegradable copolymer according to claim 29 in the manufacture of a medicament for the delivery of a nucleic acid molecule to a cell in an animal.
